(19) United States
(12) Patent Application Publication
(10) Pub. No.: US 2026/0274778 A1
(43) Pub. Date: Sep. 17, 2026

(54) A PROCESS FOR PREPARING 2,2-DIMETHOXYPROPANE

(71) Applicant: ADITYA BIRLA CHEMICALS (THAILAND) LIMITED, BANGKOK (TH)

(72) Inventors: Thube SANDIP, MUMBAI (IN); Bharat SINGH, MUMBAI (IN); Pankaj MATHURE, MUANG, RAYONG (TH); Szymon Tadeusz KOSINSKI, MUANG, RAYONG (TH); Pradip Kumar DUBEY, MUMBAI (IN)

(21) Appl. No.: 19/122,999

(22) PCT Filed: Oct. 21, 2023
(86) PCT No.: PCT/IB2023/060648

§ 371 Date: Apr. 21, 2025

(30) Foreign Application Priority Data
Oct. 21, 2022 (IN) 202221060664

(51) Int. Cl. C07C 41/56; C07C 41/58

(52) U.S. Cl. CPC C07C 41/56; C07C 41/58

(57) ABSTRACT

A process for preparing 2,2-dimethoxypropane includes the steps of reacting acetone with methanol in a molar ratio ranging between 1:2 to 1:6 in the presence of a catalyst at a reaction temperature in the range of −50° C. to −80° C., followed by allowing acetone and methanol to react at the reaction temperature in the range of −50° C. to −80° C. for a time period in the range of about 12 hours to 7 days to obtain a reaction mixture including crystals of 2,2-dimethoxypropane; quenching the reaction mixture including crystals of 2,2-dimethoxypropane with a base to obtain a slurry including crystals of 2,2-dimethoxypropane. The slurry including crystals of 2,2-dimethoxypropane is treated to obtain 2,2-dimethoxypropane in a liquid form.

### FIELD OF THE INVENTION

The present disclosure relates to a process for preparing 2,2-dimethoxypropane (commonly known as DMP). In particular, the present disclosure relates to a cost effective and energy efficient process for preparing 2,2-dimethoxypropane by preparation of crystals of 2,2-dimethoxypropane.

### BACKGROUND

2,2-dimethoxypropane, an organic compound, has several uses, including but not limited to as a water scavenger in water-sensitive reactions; as an intermediate for the synthesis of 2-methoxypropene; for production of insecticides and fungicides; for the dehydration of animal tissue in histology; and for the manufacturing of 2-methoxypropene for vitamins D and E.

2,2-dimethoxypropane is the condensation product of acetone and methanol, prepared in the presence of an acid catalyst. However, the reaction is reversible and at ambient temperature or above, the equilibrium of the reaction is shifted to the side of the starting materials. As the equilibrium is favored towards the starting materials, it is required to shift the equilibrium towards 2,2-dimethoxypropane by using low temperatures, high methanol/acetone ratio and tedious pervaporation techniques to remove water and shift equilibrium to increase the yield of 2,2-dimethoxypropane. Despite this most of the prior art processes provide less than desired conversion of 2,2-dimethoxypropane from the reactants. Further, the separation or isolation of 2,2-dimethoxypropane in these processes is tedious due to the azeotropes of the components of the reaction mixture. The prior art processes do not effectively traverse the azeotropic mixtures limitations.

U.S. Pat. Nos. 9,309,177 and 9,309,178 disclose a process for preparing 2,2-dimethoxypropane, wherein the process involves reacting a ketone with an alcohol in the presence of a solid acid at a reaction temperature of below −40° C. to form a reaction product mixture, and subsequently removing water and methanol from the reaction product mixture by pervaporation using a hydrophilic membrane.

In an alternate process, it is known to use large amounts of a desiccant, calcium sulfate to shift the equilibrium in a process for preparing 2,2-dimethoxypropane. The calcium sulfate removes the water produced in the reaction. However, this would mean additional expenses to replace or regenerate the spent calcium sulfate.

U.S. Pat. No. 4,775,447 discloses conducting the process of preparation of 2,2-dimethoxypropane by reacting stoichiometric ratios of acetone and methanol, in the range of 1:1 to 1:3 moles in the presence of a strong acid-based ion-exchange resin at ambient temperature. Thereafter, the 2,2-dimethoxypropane is recovered as an azeotrope with the methanol. However, the 2,2-dimethoxypropane conversion at ambient temperature is very poor, with less than 6-8% concentration of 2,2-dimethoxypropane obtained. Further, the subsequent distillation steps, are not only cost-intensive but also require undesirably large quantities of acetone to isolate the 2,2-dimethoxypropane.

### SUMMARY

A process for preparing 2,2-dimethoxypropane is disclosed. Said process comprises the steps of reacting acetone with methanol in a molar ratio ranging between 1:2 to 1:6 in the presence of a catalyst at a reaction temperature in the range of −50° C. to −80° C., followed by allowing acetone and methanol to react at the reaction temperature in the range of −50° C. to −80° C. for a time period in the range of about 12 hours to 7 days to obtain a reaction mixture comprising crystals of 2,2-dimethoxypropane; quenching the reaction mixture comprising crystals of 2,2-dimethoxypropane with a base to obtain a slurry including crystals of 2,2-dimethoxypropane; and treating the slurry including crystals of 2,2-dimethoxypropane to obtain 2,2-dimethoxypropane in a liquid form.

### BRIEF DESCRIPTION OF DRAWINGS

FIG. 1 depicts the effect of different reaction time on the conversion of 2,2-dimethoxypropane in accordance with an embodiment of the present disclosure.

FIG. 2 depicts the effect of different reaction temperatures on the conversion of 2,2-dimethoxypropane in accordance with an embodiment of the present disclosure.

FIG. 3 depicts the formation of white crystals of 2,2-dimethoxypropane in an intermediate step in accordance with an embodiment of the present disclosure.

FIGS. 4a and 4b depict the formation of white crystals of 2,2-dimethoxypropane after the quenching of the reaction mixture in accordance with an embodiment of the present disclosure.

### DETAILED DESCRIPTION

To promote an understanding of the principles of the disclosure, reference will now be made to embodiments and specific language will be used to describe the same. It will nevertheless be understood that no limitation of the scope of the disclosure is thereby intended, such alterations and further modifications in the disclosed composition and process, and such further applications of the principles of the disclosure therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “one embodiment” “an embodiment” or similar language means that a particular feature, structure, or characteristic described in connection with the embodiment is included in at least one embodiment of the present disclosure. Thus, appearances of the phrase “in one embodiment”, “in an embodiment” and similar language throughout this specification may, but do not necessarily, all refer to the same embodiment.

In the broadest scope, the present disclosure relates to a process for preparing 2,2-dimethoxypropane. Specifically, the disclosed process comprises the steps of:
reacting acetone with methanol in a molar ratio ranging between 1:2 to 1:6 in the presence of a catalyst at a reaction temperature in the range of −50° C. to −80° C.;
allowing acetone and methanol to react at the reaction temperature in the range of −50° C. to −80° C. for a time period in the range of about 12 hours to 7 days to obtain a reaction mixture comprising crystals of 2,2-dimethoxypropane;
quenching the reaction mixture comprising crystals of 2,2-dimethoxypropane with a base to obtain a slurry including crystals of 2,2-dimethoxypropane; and
treating the slurry including crystals of 2,2-dimethoxypropane to obtain 2,2-dimethoxypropane in a liquid form.

The present inventors found that the disclosed process resulting in the formation of crystals, increases the conversion of 2,2-dimethoxypropane to about 60-70%.

In an embodiment, acetone is reacted with methanol in the molar ratio of 1:2 to 1:4. In some embodiments, acetone is reacted with methanol in the molar ratio of 1:4.

In the disclosed process, acetone is reacted with methanol at a very low reaction temperature. In an embodiment, the reaction of acetone with methanol is carried out at the reaction temperature in the range of −60 to −65° C. In some embodiments, the reaction of acetone with methanol is carried out at −65° C.

In an embodiment, the reaction of acetone with methanol is carried out under a nitrogen (N₂) atmosphere under 1 bar pressure.

Once the reaction of acetone and methanol has been initiated, the formation of the 2,2-dimethoxypropane in the reaction mixture initiates at least 24 hours of initiation of the reaction. In an embodiment, acetone is allowed to react with methanol for a time period of 24 hours to 72 hours to obtain the reaction mixture comprising crystals of 2,2-dimethoxypropane. In some embodiments, acetone is allowed to react with methanol for a time period of 48 hours.

In an embodiment, the formation of the crystals of 2,2-dimethoxypropane takes place at a temperature in the range of −60 to −75° C. In some embodiments, the formation of crystals of 2,2-dimethoxypropane takes place at −65° C.

In an embodiment, acetone is reacted with methanol without stirring. In an alternate embodiment, acetone is reacted with methanol under continuous stirring at a stirring rate of 40-200 rpm.

In the next step, the reaction mixture comprising crystals of 2,2-dimethoxypropane is quenched with the base at a temperature −50° C. to −80° C. to obtain the slurry including crystals of 2,2-dimethoxypropane. In an embodiment, the reaction mixture comprising crystals of 2,2-dimethoxypropane is quenched with the base for a time period in the range of 1 minute to 1 hour. In some embodiment, the quenching of the reaction mixture with the base is carried out at −65° C. for 5 to 15 minutes.

In an embodiment, the base for quenching of the reaction mixture comprising crystals of 2,2-dimethoxypropane is selected from the group consisting of triethylamine, sodium hydroxide, sodium carbonate, potassium hydroxide, sodium methoxide, triethanolamine, and trimethylamine. In some embodiments, the base is triethylamine. In an embodiment, the base is added in a w/w ratio ranging from 0.5 to 5% to the reaction mixture comprising crystals of 2,2-dimethoxypropane. In some embodiments, the base is added in a 1-2% w/w to the reaction mixture comprising crystals of 2,2-dimethoxypropane.

The slurry including crystals of 2,2-dimethoxypropane is treated to obtain the liquid form of 2,2-dimethoxypropane. In an embodiment, the treatment of the slurry comprises filtration of the slurry to recover purified crystals of 2,2-dimethoxypropane therefrom and a filtrate. In an embodiment, the slurry including crystals of 2,2-dimethoxypropane is filtered at a temperature in the range of −60° C. to −75° C. to recover the purified crystals of 2,2-dimethoxypropane. In some embodiments, the slurry including crystals of 2,2-dimethoxypropane is filtered at −60° C. to −65° C. to recover the purified crystals of 2,2-dimethoxypropane. The filtration is carried out using any method now known or developed in the future. In some embodiments, filtration is carried out by pre-cooled monoplate filter. In the next step, the purified crystals of 2,2-dimethoxypropane are allowed to melt by warming up above −55° C. to obtain 2,2-dimethoxypropane in the liquid form. In an embodiment, the purified crystals of 2,2-dimethoxypropane are allowed to melt by allowing the crystals of 2,2-dimethoxypropane to reach room temperature.

In an embodiment, after the recovery of the purified crystals of 2,2-dimethoxypropane from the slurry, the filtrate is subjected to a recycling step to recover one or more of acetone, methanol, and water. The recycling is done using any process known in the art or developed in the future.

In an embodiment, the filtrate is recycled by a distillation method. Examples of distillation method include, but are not limited to simple distillation, fractional distillation, steam distillation, distillation under vacuum, and zone distillation.

In an alternate embodiment, the treatment of the slurry for obtaining crystals of 2,2-dimethoxypropane comprises adding a solution of sodium hydroxide to the slurry including crystals of 2,2-dimethoxypropane. In an embodiment, the slurry is treated with the solution of sodium hydroxide at a temperature in the range of −20° C. to −60° C. to for a time period in the range of 0.5 to 2 hours to obtain 2,2-dimethoxypropane in the liquid form. The addition of the solution of sodium hydroxide in the slurry including crystals of 2,2-dimethoxypropane causes melting of the crystals of 2,2-dimethoxypropane to obtain the liquid form of 2,2-dimethoxypropane. In an embodiment, the liquid 2,2-dimethoxypropane is obtained as a separate top layer from a bottom basic layer of the solution of sodium hydroxide.

In an embodiment, the solution of sodium hydroxide has concentration in the range of 20-50% w/w in water. In some embodiments, the solution of sodium hydroxide has concentration of 35% w/w in water. In an embodiment, the solution of sodium hydroxide is added in a range of 50 to 500% w/w of total mass of the slurry including crystals of 2,2-dimethoxypropane. In some embodiments, the solution of sodium hydroxide is added in the range of 150-250% w/w of the total mass of the slurry including crystals of 2,2-dimethoxypropane.

In an embodiment, the catalyst is added in a w/w ratio ranging from 0.5% to 5% of combined mass of acetone and methanol. In some embodiment, the catalyst is added in 1% w/w ratio of combined mass of acetone and methanol. In an embodiment, the catalyst is selected from the group consisting of an acid ion exchange resin and an acid.

Any acid ion exchange resin known for the preparation of DMP can be used. In an embodiment, the acid ion exchange resin is selected from the group consisting of polystyrene copolymer with sulphonic acid functional groups, and styrene-divinylbenzene copolymer with sulphonic acid functional groups. In some embodiments, the acid ion exchange resin is styrene-divinylbenzene copolymer with sulphonic acid functional groups. Any acid known for the preparation of DMP can be used. In an embodiment, the acid is selected from the group consisting of sulfuric acid, hydrochloric acid, nitric acid, para-toluenesulfonic acid, methanesulfonic acid, phosphoric acid, and acetic acid. In some embodiments, the acid is sulfuric acid.

In an embodiment, productivity of the liquid form of 2, 2-dimethoxypropane obtained from the disclosed process is in the range of 20-40% of total reaction mass.

The invention will now be described with respect to the following examples which do not limit the disclosed method in any way and only exemplify the claimed method. It will be apparent to those skilled in the art that various modifications and variations can be made to the method/process of the present disclosure without departing from the scope of the disclosure. Other embodiments will be apparent to those skilled in the art from consideration of the specification and practice of the method/process disclosed herein. It is intended that the specification and examples be considered as exemplary only, with a true scope of the disclosure being indicated by the following claims and their equivalent.

### EXAMPLES

### Example 1: Preparation of 2,2-Dimethoxypropane in Accordance with an Embodiment of the Present Disclosure

58 gm of acetone, 128 gm of methanol (mole ratio 1:4), and 10 gm of Tulsion T-62MP were charged in a jacketed reactor to form a reaction mixture. The reactor including the reaction mixture was cooled to a temperature of −60° C. to −65° C. An arrangement was made for the blanketing of the reactor with liquid nitrogen cooling jacket to maintain a low temperature for the reaction. After commencement of the reaction, in the first 24 hours, reaction goes to up to 40% conversion as determined by the Gas Chromatography (GC) analysis. After 24 hours of the reaction of acetone methanol, the formation of crystals of 2,2-dimethoxypropane began and the reaction mixture comprising crystals of 2,2-dimethoxypropane was obtained. The process of formation of crystals of 2,2-dimethoxypropane slowly proceeds further over the next 24 hours. The reaction mixture comprising crystals of 2,2-dimethoxypropane was quenched with 1% triethylamine to obtain a slurry including crystals of 2,2-dimethoxypropane. The slurry including crystals of 2,2-dimethoxypropane was filtered to recover the purified crystals of 2,2-dimethoxypropane. The purified crystals of 2,2-dimethoxypropane were maintained at room temperature to cause melting thereof and obtain 2,2-dimethoxypropane in the liquid form. After the recovery of the purified crystals of 2,2-dimethoxypropane from the slurry, the filtrate is subjected to a recycling step to obtain one or more of acetone, methanol, and water. Characterization of prepared 2,2-dimethoxypropane was carried out by GC analysis. Table 1 shows effect of the reaction time (obtained using GC analysis) on the conversion of 2,2-dimethoxypropane.

TABLE 1
Effect of the reaction time on the conversion of 2,2-dimethoxypropane

| | | GC Analysis (Area %) | | | |
|---|---|---|---|---|---|
| | | | | 2,2- | |
| Time | Observation | Acetone | Methanol | dimethoxypropane | Conversion |
| 6 | No | 25.93 | 47.81 | 26.24 | 47% |
| hours | Crystallization | | | | |
| | observed | | | | |
| 42 | Crystallization | 19.80 | 42.55 | 37.64 | 68% |
| hours | started after | | | | |
| | about 24 hours, | | | | |
| | and continued | | | | |
| | for additional 24 | | | | |
| | hours | | | | |

Observations: It was observed that carrying out the process using acetone and methanol in the molar ratio of 1:4 at the reaction temperature of −60° C. for a longer period of time over 24 hours results in the formation of crystals of 2, 2-dimethoxypropane. It was also observed that the conversion of 2,2-dimethoxypropane increases to 68% with the reaction time of 42 hours.

The same experiment as performed in example 1 was performed on a 5 liters' scale. The reaction of acetone with methanol in the molar ratio of 1:4 at the reaction temperature of −60° C. was carried out for a time period of 48 hrs. Table 2 summarizes the effect of the reaction time on the conversion of 2,2-dimethoxypropane.

TABLE 2
Effect of the reaction time on the conversion of 2,2-dimethoxypropane

| | GC Analysis (Weight %) | | | | |
|---|---|---|---|---|---|
| | | | 2,2- | | |
| Observations | Acetone | Methanol | dimethoxypropane | Water | Conversion |
| Crystallization | 12.5% | 48.20% | 33.50% | 5.8% | 60% |
| started after 24 | | | | | |
| hrs. and | | | | | |
| continued for | | | | | |
| additional 24 hrs. | | | | | |

Observations: It was observed that carrying out the process with the reaction time of 48 hours results in the formation of crystals of 2,2-dimethoxypropane. It was also observed that the conversion of 2,2-dimethoxypropane increases to 60% with the reaction time of 48 hrs.

FIG. 1 depicts the effect of the reaction time on the conversion at the molar ratio of 1:4 and reaction temperature of −60° C. It was observed that the conversion of,2-dimethoxypropane increases with the longer reaction time of over 40 hours.

### Example 2: Preparation of 2,2-Dimethoxypropane in Accordance with an Embodiment of the Present Disclosure

2.0 kg of acetone, 4.41 kg of methanol and 64 g of sulphuric acid were charged in a jacketed reactor to form a reaction mixture. The reactor including the reaction mixture was cooled to a temperature to −20° C. for 2 hours, −40° C. for 2 hrs, and then cooled to −60° C. to −65° C. for 48 hrs. An arrangement was made for the blanketing of the reactor with liquid nitrogen cooling jacket to maintain the low temperature. After 24 hours of the reaction of acetone methanol, the formation of crystals of 2,2-diemthoxypropane began and the reaction mixture comprising crystals of 2,2-dimethoxypropane was obtained. In the first 24 hours, reaction goes to up to 40% conversion as determined by the GC analysis. The process of crystals of 2,2-dimethoxypropane slowly proceeds further over the next 24 hours. The reaction mixture comprising crystals of 2,2-dimethoxypropane was quenched with 1% triethylamine to obtain a slurry including crystals of 2,2-dimethoxypropane. The slurry including crystals of 2,2-dimethoxypropane was filtered to recover the purified crystals of 2,2-dimethoxypropane. The purified crystals of 2,2-dimethoxypropane were maintained at room temperature to cause melting thereof and obtain 2,2-dimethoxypropane in the liquid form. After the recovery of the purified crystals of 2,2-dimethoxypropane from the slurry, the filtrate is subjected to a recycling step to obtain one or more of acetone, methanol, and water. Characterization of prepared 2,2-dimethoxypropane was carried out by GC analysis. Table 3 shows effect of the reaction time and the reaction time (obtained using GC analysis) on the conversion of 2,2-dimethoxypropane.

TABLE 3
Effect of the reaction time and the reaction temperature
on the conversion of 2,2-dimethoxypropane

| | GC Analysis (Weight %) | | | | |
|---|---|---|---|---|---|
| | | | 2,2- | | |
| Observations | Acetone | Methanol | dimethoxypropane | Water | Conversion |
| Crystallization | 9.43 | 44.82 | 39.00 | 6.75% | 69.75% |
| started after 24 | | | | | |
| hrs. and | | | | | |
| continued for | | | | | |
| additional 24 hrs. | | | | | |

Observations: It was observed that the conversion of 2,2-dimethoxypropane increases to 69.75% with the reaction time of 48 hrs.

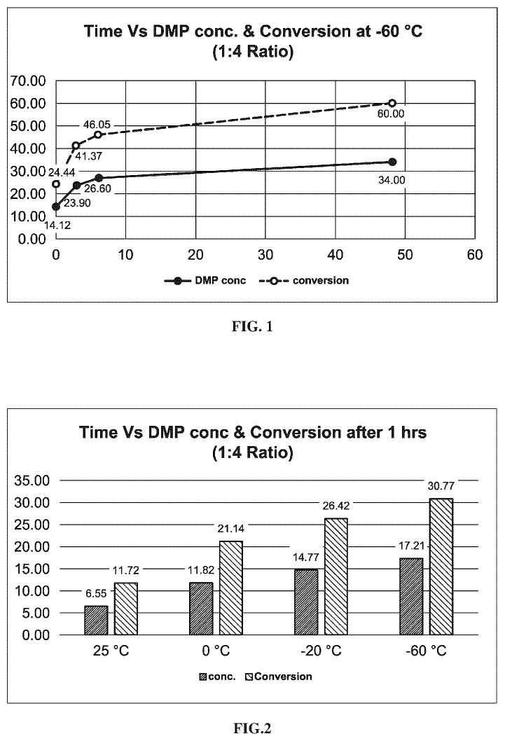

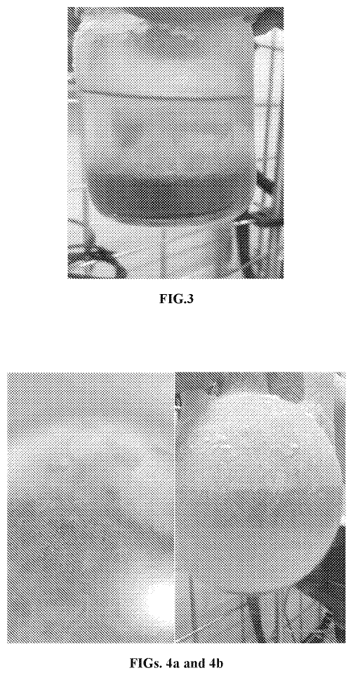

FIG. 2 depicts the effect of different reaction temperature on the conversion of 2, 2-dimethoxypropane on the reaction of acetone and methanol at the mole ratio of 1:4 for the reaction time of one hour on the conversion of 2, 2-dimethoxypropane. It was observed that the conversion of 2,2-dimethoxypropane increases with the decrease in the reaction temperature.

### Example 3: Preparation of 2,2-Dimethoxypropane According to the Processes Known in the Prior Art

2,2-dimethoxypropane was prepared by using acetone and methanol in a mole ratio of 1:1, 1:2, and 1:4. The acid catalyst resin was added in the specific amount as mentioned in Table 3 below. Different reaction temperature and reaction time was maintained during the reaction of acetone and methanol as specified in table 3 below. In the comparative examples, the reaction time duration was less than 24 hours. These are the typical conditions which are applied in the prior art processes.

TABLE 4
GC analysis of 2,2-dimethoxypropane prepared according
to the known processes of the prior art.

| | | | GC Analysis (W %) | | | | |
|---|---|---|---|---|---|---|---|
| | | | | | 2,2- | | |
| Raw material | Temperature | | Acetone | Methanol | dimethoxypropane | Conversion | Water |
| Comparative | Room | | 61.66 | 32.48 | 4.99 | 4.32 | 0.86 |
| example 1: | temperature | | | | | | |
| 10 g | 0° | C. | 59.46 | 30.06 | 8.93 | 7.73 | 1.55 |
| acetone + 5.5 | −20° | C. | 57.95 | 28.39 | 11.65 | 10.08 | 2.02 |
| g methanol | −60° C. for 5 | | 56.57 | 26.87 | 14.12 | 12.22 | 2.44 |
| (1:1) + 0.5 gm | minutes | | | | | | |
| Ion Exchange | −60° C. for 1 | | 55.52 | 25.71 | 16.00 | 13.85 | 2.77 |
| Resin in acidic | hour | | | | | | |
| form. | −60° C. for 4 | | 53.89 | 23.91 | 18.92 | 16.38 | 3.28 |
| | hours | | | | | | |
| Comparative | Room | | 44.02 | 48.57 | 6.32 | 7.41 | 1.09 |
| example 2: | temperature | | | | | | |
| 10 gm of | 0° | C. | 40.32 | 44.49 | 12.95 | 15.19 | 2.24 |
| Acetone, | −20° | C. | 39.22 | 43.28 | 14.92 | 17.51 | 2.58 |
| 11.03 gm | −60° C. for 5 | | 38.27 | 42.23 | 16.62 | 19.50 | 2.88 |
| methanol | minutes | | | | | | |
| (1:2), and 0.5 | −60° C. for 1 | | 37.36 | 41.22 | 18.26 | 21.42 | 3.16 |
| gm ion | hr. | | | | | | |
| exchange resin | −60° C. for 4 | | 35.71 | 39.40 | 21.21 | 24.89 | 3.67 |
| in acidic form. | hrs. | | | | | | |
| Comparative | Room | | 27.53 | 64.79 | 6.55 | 11.72 | 1.13 |
| example 3: | temperature | | | | | | |
| 5 g acetone, | 0° | C. | 24.59 | 61.54 | 11.82 | 21.14 | 2.05 |
| 5.5 g methanol | −20 | C. | 22.94 | 59.73 | 14.77 | 26.42 | 2.56 |

| | | | | | | |
|---|---|---|---|---|---|---|
| (1:2) and | −60° C. for 5 | 21.59 | 58.23 | 17.21 | 30.77 | 2.98 |
| 0.5 gm ion | minutes | | | | | |
| exchange resin | −60° C. for 1 | 20.48 | 57.01 | 19.19 | 34.33 | 3.32 |
| in acidic form. | hr. | | | | | |
| | −60° C. for 4 | 19.64 | 56.08 | 20.70 | 37.02 | 3.58 |
| | hrs. | | | | | |

Observations: It was observed that the conversion to 2,2-methoxypropane was lower at room temperature, 0° C., 20° C. when compared to −60° C. for 1:1, 1:2 and 1:4 of acetone and methanol. Acetone and methanol at mole ratio of 1:4 showed the best conversion at −60° C. Table 5 below shows that the reaction of acetone with methanol having mole ratio of 1:4 at −20° C. at 1 hour and 12 hours.

TABLE 5
Effect of the reaction time on the conversion of 2,2-dimethoxypropane

| | | GC Analysis (w %) | | | | |
|---|---|---|---|---|---|---|
| | | | | 2,2- | | |
| Temperature | Time | Acetone | Methanol | dimethoxypropane | Water | Conversion |
| −20° C. | 1 | 21.67 | 58.32 | 17.05 | 2.95 | 30.49 |
| | hour | | | | | |
| | 12 | 21.64 | 58.28 | 17.12 | 2.96 | 30.62 |
| | hours | | | | | |

Observations: It was observed that at 1 hour the conversion to 2,2-dimethoxypropane was 30.49% and at 12 hours, the conversion to 2,2-dimethoxypropane was 30.62%. There was negligible increase in conversion to 2,2-dimethoxypropane when the reaction time was changed from 1 hour to 12 hours.

### INDUSTRIAL APPLICABILITY

The present disclosure provides an efficient, simple and highly productive process for the preparation of 2,2-methoxypropane. Said process allows to obtain more than 60% conversion of 2,2-dimethoxypropane in one operation.

The disclosed process allows elimination of multiple heating, cooling operations, and multiple stages of pervaporation, required to achieve high conversion of 2,2-dimethoxypropane. Thus, the disclosed process overcomes the disadvantages, such as-high carbon dioxide emissions, and high production cost, associated with the pervaporation and rectification steps used in the prior art.

1. A process for preparing 2,2-dimethoxypropane, the process comprising the steps of:
reacting acetone with methanol in a molar ratio ranging between 1:2 to 1:6 in the presence of a catalyst, at a reaction temperature in the range of −50° C. to −80° C.;
allowing acetone and methanol to react at the reaction temperature in the range of −50° C. to −80° C. for a time period in the range of about 12 hours to 7 days to obtain a reaction mixture comprising crystals of 2,2-dimethoxypropane;
quenching the reaction mixture comprising crystals of 2,2-dimethoxypropane with a base to obtain a slurry including crystals of 2,2-dimethoxypropane; and
treating the slurry including crystals of 2,2-dimethoxypropane to obtain 2,2-dimethoxypropane in a liquid form.

2. The process as claimed in claim 1, wherein acetone and methanol are reacted in a molar ratio of 1:4.

3. The process as claimed in claim 1, wherein the reaction of acetone with methanol is carried out at the reaction temperature of −60° C. to −65° C.

4. The process as claimed in claim 1, wherein the treatment of the slurry comprises filtering the slurry to recover purified crystals of 2,2-dimethoxypropane therefrom and a filtrate, followed by melting of the purified crystals of 2,2-dimethoxypropane to obtain 2,2-dimethoxypropane in the liquid form.

5. The process as claimed in claim 4, wherein after the recovery of the purified crystals of 2,2-dimethoxypropane from the slurry, the filtrate is subjected to a recycling step to obtain one or more of acetone, methanol, and water.

6. The process as claimed in claim 1, wherein the treatment of the slurry comprises adding a solution of sodium hydroxide to the slurry containing crystals of 2,2-dimethoxypropane to obtain 2,2-dimethoxypropane in the liquid form as a separate top layer.

7. The process as claimed in claim 6, wherein the solution of sodium hydroxide has concentration of 20-50% in water and is added in a range of 50 to 500% w/w of total mass of the slurry containing crystals of 2,2-dimethoxypropane.

8. The process as claimed in claim 1, wherein the catalyst is added in a w/w ratio ranging from 0.5% to 5% of combined mass of acetone and methanol.

9. The process as claimed in claim 1, wherein the catalyst is selected from the group consisting of an acid ion exchange resin and an acid.

10. The process as claimed in claim 1, wherein the catalyst is the acid ion exchange resin selected from the group consisting of polystyrene copolymer with sulphonic acid functional groups, and styrene-divinylbenzene copolymer with sulphonic acid functional groups.

11. The process as claimed in claim 9, wherein catalyst is the acid selected from the group consisting of sulfuric acid, hydrochloric acid, nitric acid, para-toluenesulfonic acid, methanesulfonic acid, phosphoric acid, and acetic acid.

12. The process as claimed in claim 1, wherein acetone and methanol are reacted without stirring.

13. The process as claimed in claim 1, wherein the quenching of the reaction mixture with the base is carried out at a temperature in the range of −50° C. to −80° C. for a time period in the range of 1 minute to 1 hour.

14. The process as claimed in claim 1, wherein the base is selected from the group consisting of triethylamine, sodium hydroxide, sodium carbonate, potassium hydroxide, sodium methoxide, triethanolamine, and trimethylamine.

15. The process as claimed in claim 13, wherein the base is added in a w/w ratio ranging from 0.5 to 5% to the reaction mixture containing crystals of 2,2-dimethoxypropane.
